(19) United States
(12) Patent Application Publication
(10) Pub. No.: US 2015/0165150 A1
(43) Pub. Date: Jun. 18, 2015

(54) Respiratory Mask With Self-Sealing Implement Port

(71) Applicant: Engineered Medical Systems, Inc., Indianapolis, IN (US)

(72) Inventors: Brad Quinn, Indianapolis, IN (US); Bradley Allen Wheeler, Greenwood, IN (US)

(21) Appl. No.: 14/515,247

(22) Filed: Oct. 15, 2014

Related U.S. Application Data
(60) Provisional application No. 61/916,639, filed on Dec. 16, 2013.

(51) Int. Cl. A61M 16/06; A61J 15/00

(52) U.S. Cl. CPC A61M 16/0605; A61J 15/0057; A61M 2016/0661; A61M 2205/05

(57) ABSTRACT

A respiratory mask for delivery of pressurized gas to a patient. The respiratory mask has a rigid shield and an elastic cushion surrounding the shield, the cushion being configured to conform to facial contours of a patient. A port is provided in the cushion, the port being configured to receive an implement inserted into the port and to provide a substantially gas-tight seal about the periphery of an implement received in the port. The port may be located in an area of the cushion that allows for a relatively direct route to the nares of the patient of the patient to allow insertion of the NG tube into the patient's nares. Embodiments may include the port being a slit in the elastic cushion.

### RELATED APPLICATIONS

This application claims priority from U.S. Provisional Patent Application Ser. No. 61/916,639, filed Dec. 16, 2013, entitled “Respiratory Mask With Self-Sealing Implement Port,” which is hereby incorporated by reference.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### TECHNICAL FIELD

Respiratory masks and more particularly, non-invasive ventilation masks having a port for receiving an implement such as a nasogastric tube for use by a patient.

### BACKGROUND

Various respiratory masks are used for delivering gases to the nose and mouth of a patient. One type of respiratory masks are non-invasive ventilation (“NIV”) masks which generally comprise a rigid cone or shield for covering a portion of the patient's face and a cushion about the cone or shield which is compliant and conformable to the contours of a patient's face to form a substantially gas-tight seal with the patient's face. As used herein, “substantially gas-tight seal” means a seal of sufficient integrity that a gas can be supplied to a patient at a desired pressure without the seal diminishing the therapeutic effect of the supply of gas. It may be desirable at times to provide additional therapies to a patient using an NIV mask. For example, it may be desirable to provide nutrition to the patient by a nasogastric (“NG”) tube which is received in the nares of the patient during NIV therapy. It is known to provide a port in the cone or shield of the mask to receive an NG tube which can then be inserted into the nares of the patient. However, such ports do not provide a substantially gas-tight seal about the periphery of the NG tube and thus allow for the loss of pressurized gas from around the periphery of the NG tube which can affect the therapeutic delivery of the pressured gas to the patient. Also, such ports are typically provided at a location requiring a sharp bend in the NG tube after it is received in the port of the mask in order to insert the NG tube into the nares of the patient. This can be both difficult to manipulate during insertion of the NG tube into the nares of the patient and uncomfortable for the patient after insertion.

The present invention is directed toward overcoming one or more of the problems discussed above.

### SUMMARY OF THE EMBODIMENTS

Disclosed is a respiratory mask for delivery of pressurized gas to a patient. The respiratory mask has a rigid shield and an elastic cushion surrounding the shield, the cushion being configured to conform to facial contours of a patient. A port is provided in the cushion, the port being configured to receive an implement inserted into the port and to provide a substantially gas-tight seal about the periphery of an implement received in the port. Embodiments include the port being located in an area of the cushion that allows for a relatively direct route to the nares of the patient to allow insertion of the NG tube into the patient's nares. Embodiments may include the port being a slit in the cushion, the port having an “s” shape configured to form an orifice which substantially seals the cushion about the periphery of the implement. Embodiments may also include the port being slightly smaller than the outer circumference of the implement to allow the cushion material to stretch and form a tight seal with the periphery of the implement. Embodiments may also include a thin film over the port to seal the port prior to the insertion of an implement. In one embodiment, the film may be disposed over a port defined by a slit. In other embodiments the port may just be a hole in the cushion with the film providing the substantially gas-tight seal before insertion of an implement therein.

Another aspect of the invention is a method of providing access of an implement to a nose or mouth of a patient receiving non-invasive ventilation via a respiratory mask. The method includes providing a respiratory mask with a rigid shield and an elastic cushion surrounding the shield, the cushion being configured to conform to facial contours of a patient. An implement having a select outer diameter is provided. A slit is provided in the cushion of the mask, the slit being configured to substantially prevent the passage of gas through a gas-tight seal about the periphery of the slit. The implement is axially inserted into the slit.

Various modifications and additions can be made to the embodiments discussed without departing from the scope of the invention. For example, while the embodiments described above refer to particular features, the scope of this invention also included embodiments having different combination of features and embodiments that do not include all of the above described features.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is bottom, front perspective view of a respiratory mask having a self-sealing implement port in the mask cushion;

FIG. 2 is a bottom, plan view of the respiratory mask of FIG. 1 showing an embodiment of the self-sealing implement port;

FIG. 3 is a perspective view of the inside of an embodiment of the self-sealing implement port receiving an implement therein;

FIG. 4 is a cross-section of the self-sealing implement port of FIG. 3;

FIG. 5 is a cross-sectional view of a respiratory mask in accordance with embodiments of the invention disposed upon the face of a patient showing an NG tube received in the nares of the patient's nose.

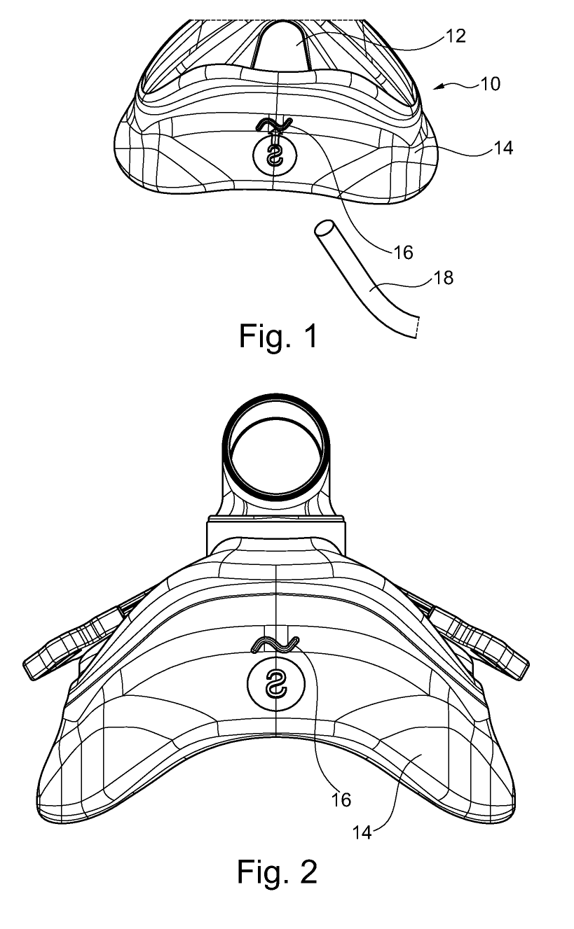

### DETAILED DESCRIPTION

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

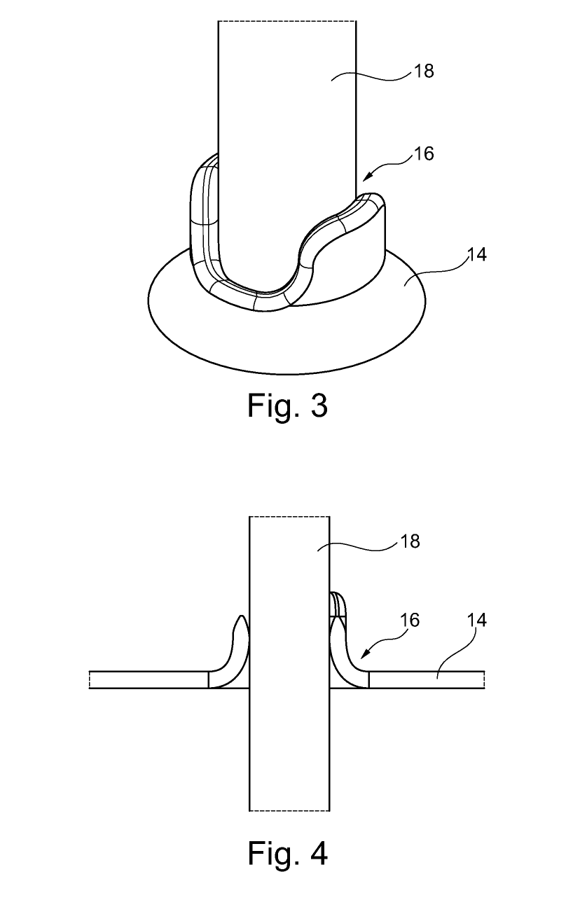

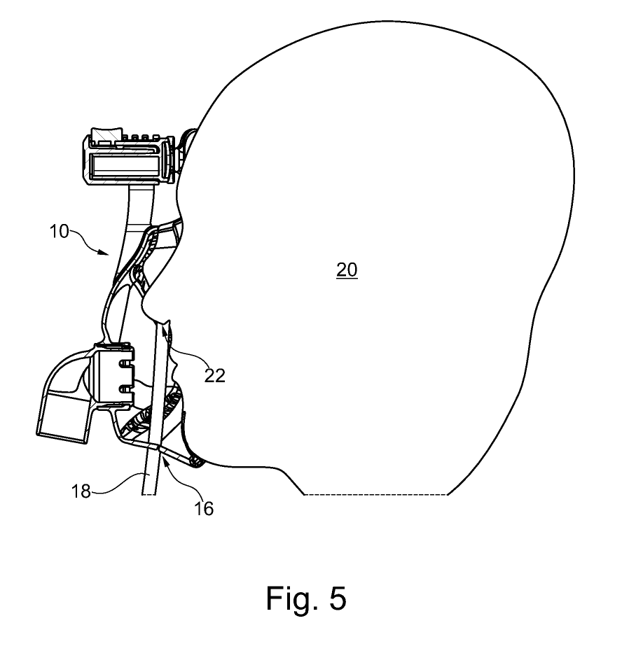

FIG. 1 is a bottom, front perspective view of a respiratory mask 10 which may be, for example, an NIV face mask. The respiratory mask 10 comprises a shield 12 formed of a rigid plastic material such as, for example, a clear polycarbonate plastic. An elastic cushion 14 surrounds the periphery of the shield 12. The elastic cushion 14 is made of a flexible, stretchable and conformable material to conform to facial contours of a patient as illustrated in part in FIG. 5. The elastic cushion 14 may be made of, for example, silicon plastic. A port 16 in the form of a slit is shown in the bottom of the elastic cushion 14. In the embodiment illustrated in FIG. 1, the slit 16 has an “s” shaped configuration sized to receive an implement 18, which may be, for example, an NG tube. Embodiments of the port 16 could also include a hole with a removable plug or slits having other configurations, such as a straight slit, an “x” shaped slit (i.e., a four-flap slit), a tri-flap slit or a slit defining more than four flaps. The essential feature of the slit 16 is that it forms a substantially air-tight seal in an undisturbed state as illustrated in FIG. 1. In addition, the slit 16 must be sized to form a substantially gas-tight seal about the periphery of the implement 18 when inserted into the slit 16. Such a substantially air-tight seal is illustrated in FIGS. 3 and 4. The conformable, elastic nature of the elastic cushion 14 causes the port opening to elastically contract about the periphery of the implement 18. In certain embodiments the seal may be achieved by the port defining an opening which would normally be smaller than the outer periphery of an implement 18 inserted therein, wherein the elastic nature of the cushion cause it to form a tight seal about the implement. Embodiments include the slit 16 being integrally formed in the elastic cushion 14.

FIG. 5 is a cross-section of the respiratory mask 10 disposed on the face of a patient 20. As illustrated in FIG. 5, the slit 16 is provided below the nares 22 of a patient 20 to allow for easy access of an implement 18 such as an NG tube into the nares 22 of a patient. As illustrated, the slit 16 is located in the bottom of the elastic cushion 14 substantially directly below the nares 22 of a patient such that the slit 16 is aligned substantially parallel to the nares 22 of a patient with the respiratory mask 10 disposed operatively on the patient 20. Embodiments may include the slit 16 in other locations of the elastic cushion 14 below the nares 22 of a patient 20 that allow for the convenient and comfortable deployment of an NG tube into the nares 22 of a patient 20.

Embodiments of the respiratory mask 10 with a port in the form of a self-sealing slit 16 may include a thin plastic film that completely seals the slit 16 before insertion of an implement 18 therein. Upon insertion of an implement 18 into the slit 16, the film is torn to allow passage of the implement 18 therethrough. Another embodiment of the port could be a hole that is sealed by a thin plastic film until the film is pierced by insertion of an implement.

Embodiments include the elastic cushion 14 being made of a silicon having a durometer in the rage of 40-80 shore A. Embodiments of the elastic cushion have a thickness that allows an implement to be readily inserted therein while having sufficient integrity to maintain the substantially gas-tight seal. Silicon with a thickness in the range of 0.01-0.1 inches is believed to be acceptable.

The respiratory mask 10 could be an NIV mask intended to deliver gas at pressures ranging from 0-50 cm of H₂O. In embodiments where the port 16 is a slit, in its un-deformed state and with an implement 18 received therein the slit is configured to maintain a substantially gas-tight seal over such a range of pressures. Embodiments may include the slit 16 having a length that is no more than an outer diameter than the implement 18.

What is claimed is:

1. A respiratory mask for administering gas to a patient comprising:
a rigid shield;
a elastic cushion surrounding the shield, the cushion being configured to conform to facial contours of a patient; and
a port in the cushion, the port being configured to receive an implement inserted into the port and to provide a substantially gas-tight seal about the periphery of an implement received in the port.

2. The respiratory mask of claim 1 wherein the port is configured to be self-sealing so that it substantially prevents the passage of gas through the port without an implement received in the port.

3. The respiratory mask of claim 1 wherein the port comprises an orifice in the cushion and removable plug, the plug being receivable in the orifice to substantially prevent the passage of gas through the port.

4. The respiratory mask of claim 2 wherein the port is integrally formed in the cushion.

5. The respiratory mask of claim 4 wherein the port comprises a slit in the cushion.

6. The respiratory mask of claim 5 wherein the slit is an “s” shape.

7. The respiratory mask of claim 1 wherein cushion is formed of a silicone plastic.

8. The respiratory mask of claim 7 wherein the mask is formed of a clear, rigid plastic.

9. The respiratory mask of claim 8 wherein the clear, rigid plastic is a polycarbonate plastic.

10. The respiratory mask of claim 1 wherein the mask is a non-invasive ventilation (NIV) mask for delivering pressurized gas to a patient.

11. The respiratory mask of claim 1 wherein the port is configured to provide a substantially gas-tight seal about the periphery of an implement at pressures ranging from 0-50 cm H₂O.

12. The respiratory mask of claim 1 wherein the port is located in the cushion at a location such that the port is below the nares of a patient with the mask disposed operatively on a patient.

13. The respiratory mask of claim 12 wherein the port is located in the cushion at a location such that the port is below the chin of a patient with the mask disposed operatively on a patient.

14. The respiratory mask of claim 12 wherein the port is located in the cushion at a location substantially aligned in parallel to the nares of a patient with the mask disposed operatively on a patient.

15. The respiratory mask of claim 1 wherein the implement is tubular and the port defines an opening of size such that a portion of the cushion defining a port opening elastically contracts about the periphery of the tubular implement.

16. The respiratory mask of claim 1 further comprising a plastic film over the port which is torn by insertion of an implement through the port.

17. A method of providing access of an implement to a nose or mouth of a patient receiving non-invasive ventilation via a respiratory mask comprising:
providing a respiratory mask having a rigid shield and an elastic cushion surrounding the shield, the cushion being configured to conform to facial contours of a patient;
providing an implement having a select outer circumference;
providing a slit in the cushion of the mask, the slit being configured to provide a substantially gas-tight seal and to allow passage of the implement therethrough while maintaining a substantially gas-tight seal about the periphery of the slit; and
inserting the implement into the slit.

18. The method of claim 17 wherein the slit has a length that is no more than an outer diameter of the implement.
